(19) United States
(12) Patent Application Publication
(10) Pub. No.: US 2002/0049403 A1
(43) Pub. Date: Apr. 25, 2002

(54) Method and apparatus for permanent vascular access for hemodialysis

(76) Inventor: Audencio Alanis, Houston, TX (US)

Correspondence Address: Hugh R. Kress, 2400 Bank One Center, 910 Travis Street, Houston, TX, 77002, US

(21) Appl. No.: 09/946,185

(22) Filed: Sep. 5, 2001

Related U.S. Application Data
(60) Provisional application No. 60/237,748, filed on Oct. 3, 2000.

(51) Int. Cl. A61F 2/06

(52) U.S. Cl. 604/8; 623/1.3; 623/1.36

(57) ABSTRACT

A method and apparatus for providing permanent arterial-venous (“AV”) access to a patient for the purposes of conducting hemodialysis procedures and the like. In one embodiment, an AV graft is provided with a venous end of relatively small diameter adapted to be introduced into a patient's superior vena cava. The opposite, arterial end of the graft has a larger diameter than the venous end, while an intermediate body portion of the graft has an even larger diameter. In one configuration, the graft is introduced using a cut-down procedure in which venous access is established at a location on the patient's arm, while the venous end of the graft is fed through the patient's vein until the distal venous end thereof becomes situated in the superior vena cava. The arterial end of the graft is then tunneled down the patient's arm to a desired arterial access site, where an anastomotic coupling is effectuated. In another embodiment, the distal venous end of the graft is introduced percutaneously through the patient's jugular vein, while the body and arterial distal end are tunneled down to a selected arterial access site. The inventive graft is preferably composed of Thoralon®, PTFE or, alternatively, of a polyurethane section serving as the venous end fused to a PTFE section serving as the arterial end.

### RELATED APPLICATION DATA

This application claims the priority of prior provisional U.S. patent application Serial No. 60/237,748 filed on Oct. 3, 2000, which application is hereby incorporated by reference herein in its entirety.

### FIELD OF THE INVENTION

The present invention relates to arterial-venous (“AV”) grafts for hemodialysis patients, and more particularly relates to a method and apparatus for establishing AV grafts in for providing permanent or semi-permanent vascular access in patients in a manner which minimizes or eliminates common complications relating to such grafts.

### BACKGROUND OF THE INVENTION

For many years, the medical procedure of hemodialysis has been employed for the treatment of patients with end-stage renal diseases (commonly referred to as kidney failure). Those of ordinary skill in the medical arts will appreciate that hemodialysis essentially involves removing blood from a patient's artery, purifying the blood by a process called dialysis (the separation of impurities in blood by means of their unequal diffusion through semipermeable membranes), adding vital substances, and returning the blood to a vein. Hemodialysis is indicated especially in patients with end-stage renal disease wherein the patients' kidneys are at such a state of failure as to be incapable of performing their normal blood purification function. In many cases, a patient may be required to undergo dialysis treatment several times per week.

In a commonly employed technique for providing arterial and venous access necessary to perform dialysis, a subcutaneous arterial-venous (“AV”) graft is introduced having one end in anastomotic (fluid) communication with an artery and another end in anastomotic communication with a vein. The graft serves essentially as a direct shunt between the patient's arterial and venous systems. Once an AV graft has been established, tapping into the graft enables blood to be drawn through the graft from the patient's artery, dialyzed in an external dialysis unit, and returned by way of the graft back into the patient's vein.

An AV graft may be situated at various locations within a patient. In one common approach, a graft is disposed in a patient's forearm or upper arm, extending between the brachial artery and the vasilic vein. The graft may be disposed either above or below the skin crease opposite the patient's elbow. At each end of the graft, entry is made into the vasculature using conventional end-to-side anastomoses.

Among the more serious complications associated with dialysis procedures relates to the fluid pressures associated with reintroduction of blood into the patient's venous system after dialysis. It is known that the venous endothelial cells making up the inner walls of veins are especially fragile and susceptible to damage as blood is propelled into the vein with large force. Over time (i.e., after some number of dialysis sessions), the damage to the venous endothelial cells will cause the vein to narrow, leading ultimately to clotting of the graft. This phenomenon is known as endothelial or neointimal hyperplasia. When such hyperplasia becomes sufficiently acute, it is necessary to relocate the AV graft. For long-term dialysis patients, fewer and fewer arterial and especially venous sites which have not been deteriorated by the effects of previous grafts will be available.

Another complication with prior art AV grafting configurations relates to the deprivation of blood to parts of the body that are extreme with respect to the location of the graft. This phenomenon is sometimes referred to as “steal syndrome,” since the presence of the graft shunted between the artery and the vein essentially steals blood from downstream of the graft. For example, if an AV graft is disposed in a patient's forearm, an appreciable amount of blood is redirected by the graft directly into the venous system before it is permitted to flow on its normal arterial course, thereby depriving the patient's hand of a normal supply of blood. This can lead to swelling and associated discomfort for the patient.

### SUMMARY OF THE INVENTION

In view of the foregoing, the present invention is directed in one aspect to a method and apparatus for obviating and/or eliminating the most common complications known to be associated with AV grafts, especially those concerning neointimal hyperplasia.

In one embodiment of the invention, a graft is provided with a long venous access end. Accessing the patient's venous system through a peripheral vein via a cut-down, or through a central venous access on the right or left jugular vein, the tip of the venous side of the graft is advanced into the patient's superior vena cava. With such an arrangement, turbulence associated with the venous inflow hitting the walls of a vein is minimized or eliminated. The kinetic force of inflowing blood dissipated within the pool of blood in the superior vena cava, and is more central to the surrounding tissue of the superior vena cava relative to that in a much narrower vein, with better laminar flow.

In one embodiment of the invention, the anatomical configuration of the graft will be a tapered AV central graft measuring approximately 2-3 mm at the venous end. The body of the graft is preferably on the order of 6-7 mm and tapered to approximately 4 mm at the arterial anastomosis end.

In one embodiment, the surgical technique for implantation consists of exposing via a cut-down one of the peripheral veins, which could be the vasilic vein or axillary vien, in a customary fashion. The central venous end of the graft is inserted into the vein through a small venotomy. Once the catheter is in position with the extreme venous end in position in the superior vena cava, the 2-3 mm end of the venous graft can be secured around the vein using a purse string suture. The body of the graft can then be tunneled under the skin and exited through a counter-incision over the selected arterial in-flow site. The 4 mm end of the graft is then anastomosed to the artery in the standard end-to-side fashion.

In an alternative embodiment, the central venous end of the graft can be placed into the superior vena cava percutaneously through either the right or left internal jugular vein as performed in the insertion of chonic hemodialysis catheters. The body of the graft can similarly be tunneled under the skin and exited through a selected native arterial site for anastomosis.

In accordance with one aspect of the invention, the material from which the inventive graft is formed may be any of a number of flexible, biocompatible materials, including, without limitation, PTFE, polyurethane, a fusion of PTFE with polyurethane, or Thoralon®. The venous side of the graft (i.e., the 2-3 mm diameter portion) may be approximately 30-40 cm in length, enough to access the superior vena cava using either of the aforementioned cut-down or percutaneous insertion techniques. The body of the graft is preferably 6-7 mm in diameter and made of PTFE, tapered down to 4 mm in diameter at the end of the arterial side of the graft where the end-to-side anasomosis is to be performed. The tapering of the graft at the arterial end to 4 mm is preferable to eliminate or minimize any possible arterial steal syndrome.

In accordance with another aspect of the invention, among the principal advantages of the method and apparatus disclosed herein is the minimization of complications of neointimal hyperplasia which can lead to blockage of the venous end of the graft in standard AV graft placement in which the venous end of the graft is anastomosed to the vein. Another advantage is that the venous end of the graft is localized in a central venous site of the superior vena cava; therefore, arterial blood flow enters the center of the vein, thereby dissipating the kinetic energy downstream in a more laminar fashion. A further advantage is the elimination of the need for a venous anastomosis; thus the implantation procedure is simplified, reducing the operative time relative to standard AV graft procedures.

### BRIEF DESCRIPTION OF THE DRAWINGS

The foregoing and other features and aspects of the present invention will be best understood with reference to the following a detailed description of a specific embodiment of the invention, when read in conjunction with the accompanying drawings, wherein:

FIG. 1 is a depiction of a prior art arterial-venous (“AV) graft implanted in a patient;

FIG. 2 is a depiction of an AV graft in accordance with one embodiment of the invention;

FIG. 3 is a depiction of the AV graft from FIG. 2 implanted in an alternative configuration; and

FIG. 4 is a depiction of the AV graft from FIGS. 2 and 3 implanted using an alternative implantation technique.

### DETAILED DESCRIPTION OF A SPECIFIC EMBODIMENT OF THE INVENTION

In the disclosure that follows, in the interest of clarity, not all features of actual implementations are described. It will of course be appreciated that in the development of any such actual implementation, as in any such project, numerous engineering, programming, and design decisions must be made to achieve the developers' specific goals and subgoals (e.g., compliance with system- and business-related constraints), which will vary from one implementation to another. Moreover, attention will necessarily be paid to proper clinical, engineering and design practices for the environment(s) in question. It will be appreciated that such a development effort might be complex and time-consuming, but would nevertheless be a routine undertaking for those of ordinary skill in the elevant fields.

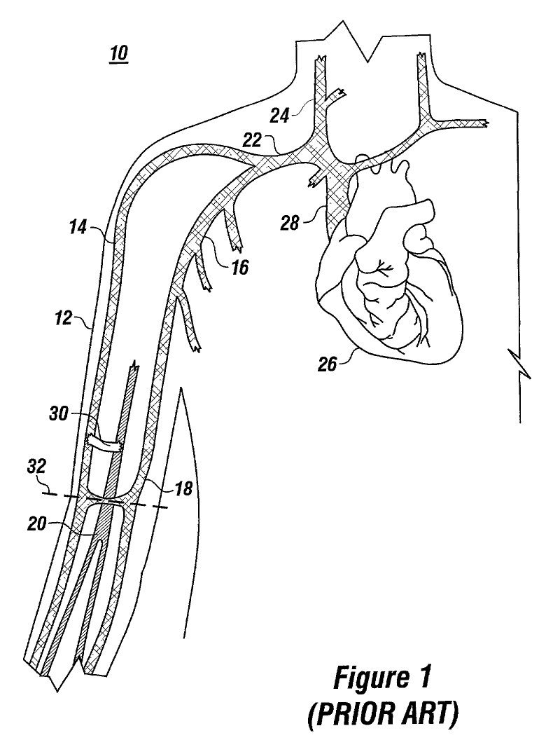

Referring to FIG. 1, there is shown a depiction of a patient's body 10 including certain anatomical features of relevance to the present invention. In particular, there is shown a portion of the patient's arm 12 and certain vasculature therein, including the patient's cephalic vein 14, axillary vein 16, vasillic vein 18, and brachial artery 20. The cephalic vein 14 extends up to the patient's subclavian vein 22, which merges with the internal jugular vein 24. The patient's heart 26 is shown, with the superior vena cava 28 disposed generally at the merger of the subclavian vein 22 and internal jugular vein 24. Those of ordinary skill in the art will appreciate that the superior vena cava 28 is the second largest vein in the human body, is formed by the union of the two brachiocephalic veins at the level of the space between the first two ribs, and returns blood to the right atrium of the heart from the upper half of the body.

FIG. 1 depicts an arterial-venous (“AV”) graft 30 extending between the patient's cephalic vein 14 and brachial artery 20 in a conventional manner. As is known in the art, such a graft 30 may be disposed above or below the patient's skin crease (generally opposite the patient's elbow) designated generally by dashed line 32 in FIG. 1. The known problems associated with such an arrangement have been discussed above.

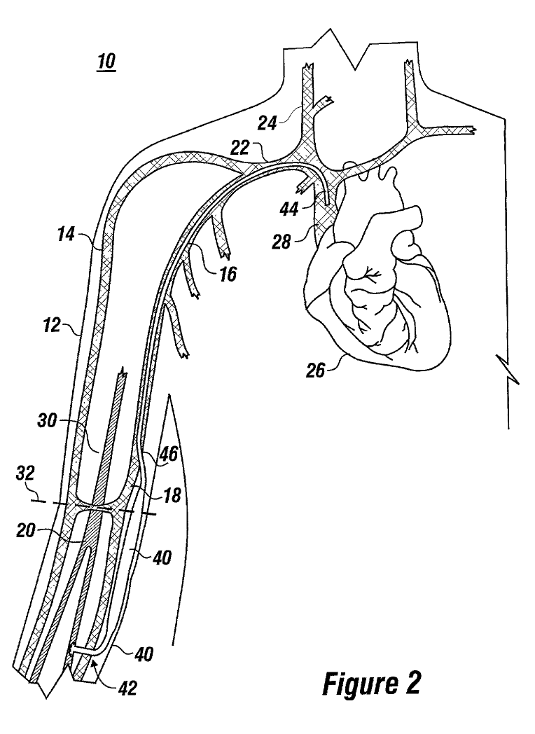

Referring to FIG. 2, there is shown an AV graft 40 in accordance with one embodiment of the invention. As can be seen from FIG. 2, graft 40 comprises an elongate, substantially tubular structure having an arterial end 42 adapted for anastomotic connection to a patient's artery, and a venous end 44 adapted to be disposed generally in a patient's superior vena cava 28. When so disposed, graft 40 establishes a shunt path between the patient's arterial and venous systems. For the purposes of dialysis, blood can be extracted from the patient by accessing the graft 40, for example, using a needle, rather than either the patent's veins or arteries directly. Advantageously, this minimizes the undesirable effects of repeated vascular access, especially in long-term dialysis patients.

After extracted blood is dialyzed using well-known conventional techniques, it may be reintroduced into the patient's vascular system via the graft 40, again, for example with a needle or the like. Advantageously, the blood enters the patient's venous system in the area of the superior vena cava, which is substantially less susceptible to damage from the in-rush of blood than are the more delicate veins.

In accordance with one feature of the invention, the dimensions of graft 40 are not uniform along its length. In particular, it is contemplated in one embodiment that graft 40 has a substantially narrower diameter at venous end 44 relative to a central “body” portion thereof, and further that the arterial end 42 will have a diameter somewhat larger than that of venous end 44, but nonetheless somewhat smaller than the central body portion. In one embodiment, it is contemplated that venous end 44 may have an outer diameter on the order of 2 to 3 millimeters, the arterial end 42 has an outer diameter of 4 millimeters, and a central body portion has a diameter of 6 to 7 millimeters. Those of ordinary skill in the art will appreciate that the foregoing approximate dimensions of graft 40 may not be ideal for all patients. Depending on a patient's physical size—for example, depending upon whether a patient is male or female, a child or an adult—different dimensions may be appropriate. In more general terms, the present invention contemplates graft 40 having scaled dimensions wherein its central body section has a diameter roughly two to three times that of venous end 44, and roughly one-and-a-half to two times that of arterial end. In general terms, it is contemplated that arterial end 42 will be somewhat larger than venous end 44 and that the central section will be somewhat larger than both arterial end 42 and venous end 44.

In one embodiment, graft 40 is composed of PTFE material, which is known to have the appropriate characteristics of biocompatibility, durability, and flexibility to serve the purposes of an AV graft. In another embodiment of the invention, graft 40 is composed partially of PTFE, but wherein a substantial portion of venous end 44 is composed of polyurethane fused to the PTFE arterial end 42 and central body section. In still another embodiment of the invention, graft 40 is made of Thoralon®, a proprietary biocompatible material produced by Thoratec Corporation, Pleasanton, Calif.

Graft 40 in FIG. 2 is shown in a “bridge” configuration. Implantation of graft 40 in the configuration begins with establishing venous access at a “cut-down” area designated generally with reference numeral 46. The cut-down area may be located in the vasillic or axillary vein. The venous end 44 of the graft 40 is inserted into the vein through a small venotomy. The distal venous end 44 of graft 40 is fed through the vein until it is disposed in the superior vena cava 28. Once the graft 40 is in position, it may be secured around the vein using a conventional purse-string suture.

Next, the body and arterial end 42 of the graft 40 are tunneled under the skin and exited through a counter-incision over the selected arterial site designated generally with reference numeral 48 in FIG. 2. The arterial end 42 is then anastomosed to the artery 20 in a conventional end-to-side fashion.

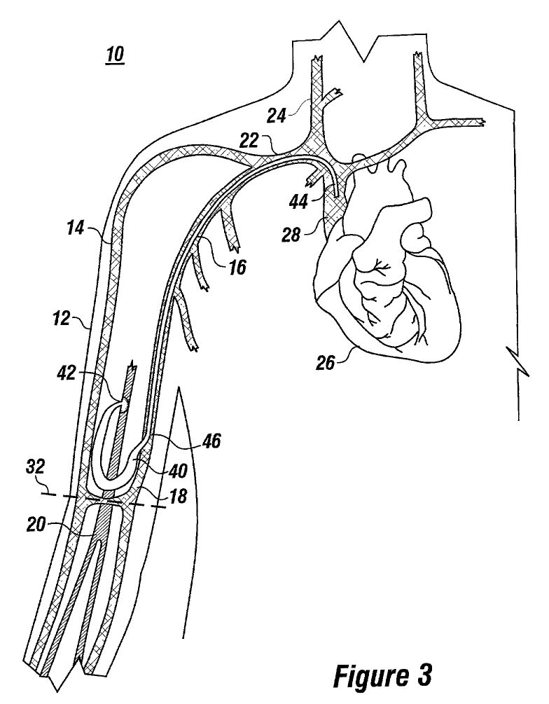

FIG. 3 shows an alternative configuration of graft 40, in which arterial end 42 is looped upward to meet artery 20 higher up on the patient's forearm. The implantation procedure for this configuration is substantially as described above.

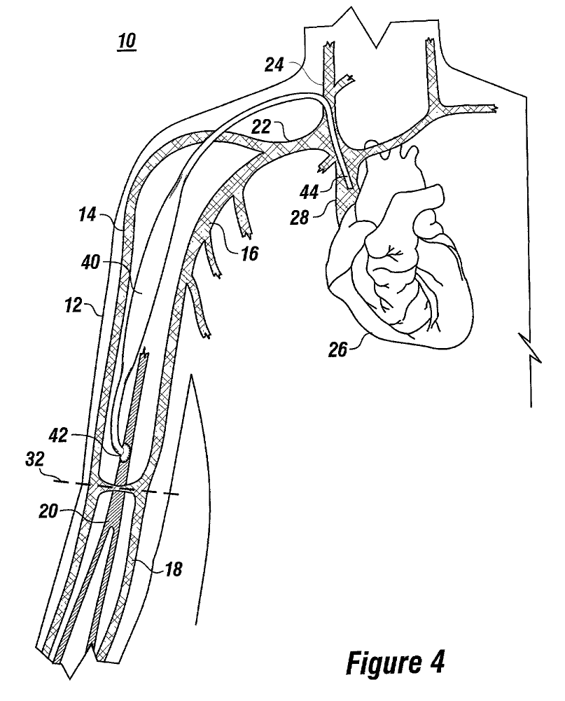

Turning to FIG. 4, there is shown another alternative configuration of graft 40. In the configuration of FIG. 4, graft 40 is implanted using a percutaneous technique. In the embodiment of FIG. 4, venous end 44 of graft 40 is inserted into superior vena cava 28 via percutaneous insertion through the patients jugular vein 24. The insertion procedure is generally as follows: First, a percutaneous needle punctures the patient's jugular vein to provide venous access. A guide wire is then inserted through the needle, and the needle is removed. Next, a peel-away introducer is threaded over the guide wire and advanced into the insertion site. The venous end 44 of graft 40 is then inserted through the introducer until the distal venous end of graft 40 becomes situated at the desired location in the superior vena cava. The introducer is then backed out and peeled away from the body of graft 40 leaving distal end 44 in its desired location.

Following insertion of the venous end 44, the body and arterial end 42 of graft 40 are tunneled down the patient's arm to the desired arterial site, where an end-to-side anastomosis coupling is achieved using conventional techniques.

The percutaneous configuration of FIG. 4 is believed to be especially indicated in situations where a patient's subclavian vein 22 exhibits the stenotic effects of repeated percutaneous access. As will be appreciated by those of ordinary skill in the art, subclavian vein 22 may be stenosed to such an extent that even the relatively small 2-3 mm diameter of venous end 44 of graft 40 cannot be inserted through to access superior vena cava 28.

From the foregoing detailed description of a specific embodiment of the invention, it should be apparent that a method and apparatus for permanent vascular access has been disclosed. Although a specific embodiment of the invention has been disclosed herein in some detail, this has been done solely for the purposes of illustrating various aspects and features of the invention, and is not intended to be limiting with respect to the scope of the invention. It is contemplated that various substitutions, alterations, modifications and/or additions, including but not limited to those design alternatives which might have been specifically noted in this disclosure, may be made to the disclosed embodiment without departing from the spirit and scope of the invention as defined in the claims which follow.

What is claimed is:

1. An arterial-venous graft, comprising a flexible elongate tubular body section, an arterial end portion, and a venous end portion, wherein said arterial end portion is adapted for anastomotic connection to a patient's artery and said venous end portion is adapted to be introduced into the superior vena cava of a patient's heart, thereby establishing a shunt path between said patient's arterial and venous systems.

2. An arterial-venous graft in accordance with claim 1, wherein said elongate tubular body section has a diameter larger than said arterial end portion and said venous end portion.

3. An arterial-venous graft in accordance with claim 2, wherein said elongate tubular body section has a diameter two to three times the diameter of said venous end portion.

4. An arterial-venous graft in accordance with claim 3, wherein said elongate tubular body section has a diameters one-and-a-half to two times larger than said arterial end portion.

5. An arterial-venous graft in accordance with claim 2, wherein said elongate tubular body section has a diameter of about six to seven millimeters.

6. An arterial-venous graft in accordance with claim 5, wherein said venous end portion has a diameter of about two to three millimeters.

7. An arterial-venous graft in accordance with claim 6, wherein said arterial end portion has a diameter of about four millimeters.

8. An arterial-venous graft in accordance with claim 2, wherein said graft is made of PTFE.

9. An arterial-venous graft in accordance with claim 2, wherein said venous end portion and central body section are made of PTFE and wherein said venous end portion is made of another material fused to said central body section.

10. An arterial-venous graft in accordance with claim 9, wherein said another material is polyurethane.

11. An arterial-venous graft in accordance with claim 2, wherein said graft is made of Thoralon®.

12. A method of establishing a shunt path between an arterial system and a venous system of a patient, comprising:
(a) anastomotically coupling an arterial end of an elongate, substantially tubular graft to an artery of said arterial system; and
(b) disposing a venous end of said graft in a superior vena cava of said patient's heart.

13. A method in accordance with claim 13, wherein said step (b) of disposing a venous end of said graft in a superior vena cava comprises inserting said venous end of said graft through a venotomy into a vein and advancing said venous end of said graft into said vein until said venous end is disposed in said superior vena cava.

14. A method in accordance with claim 14, wherein said step (a) of anastomotically coupling an arterial end of said graft comprises establishing an end-to-side anastomosis between said arterial end of said graft and an artery.

15. A method in accordance with claim 14, wherein said vein is selected from one of a cephalic vein, an axillary vein, and a vasillic vein.

16. A method in accordance with claim 15, wherein said artery is a brachial artery.

17. A method in accordance with claim 13, wherein said step (b) of disposing a venous end of said graft in a superior vena cava comprises percutaneous insertion of said venous end into a vein.

18. A method in accordance with claim 17, wherein said vein is a jugular vein.

19. A method of performing dialysis on a patient having an arterial system and a venous system including a superior vena cava, comprising:
(a) anastomotically coupling an arterial end of an elongate, substantially tubular graft to an artery of said arterial system;
(b) disposing a venous end of said graft in said superior vena cava such that a shunt path is established between said arterial system and said venous system;
(c) extracting blood from said shunt path;
(d) purifying said extracted blood; and
(e) reintroducing said purified blood into said shunt path.

20. A method in accordance with claim 19, wherein said step (b) of disposing a venous end of said graft in a superior vena cava comprises inserting said venous end of said graft through a venotomy into a vein and advancing said venous end of said graft into said vein until said venous end is disposed in said superior vena cava.

21. A method in accordance with claim 20, wherein said step (a) of anastomotically coupling an arterial end of said graft comprises establishing an end-to-side anastomosis between said arterial end of said graft and an artery.

22. A method in accordance with claim 21, wherein said vein is selected from one of a cephalic vein, an axillary vein, and a vasillic vein.

23. A method in accordance with claim 22, wherein said artery is a brachial artery.

24. A method in accordance with claim 19, wherein said step (b) of disposing a venous end of said graft in a superior vena cava comprises percutaneous insertion of said venous end into a vein.

25. A method in accordance with claim 24, wherein said vein is a jugular vein.
